(19) United States
(12) Patent Application Publication
(10) Pub. No.: US 2020/0188111 A1
(43) Pub. Date: Jun. 18, 2020

(54) HEART VALVE REPAIR

(71) Applicant: Medtronic Vascular, Inc., Santa Rosa, CA (US)

(72) Inventors: Olivia Metcalf, Santa Rosa, CA (US); Matthew E. Genovese, Windsor, CA (US); Caitlin Dorff, Santa Rosa, CA (US); Emily Grimm, Petaluma, CA (US); Fatemeh Fatemi Far, Santa Rosa, CA (US); Karan Punga, San Rafael, CA (US)

(21) Appl. No.: 16/711,119

(22) Filed: Dec. 11, 2019

Related U.S. Application Data
(60) Provisional application No. 62/779,294, filed on Dec. 13, 2018.

(51) Int. Cl. A61F 2/24

(52) U.S. Cl. CPC A61F 2/2445; A61F 2/2448; A61F 2/2466; A61B 2017/00575; A61F 2220/0016; A61F 2220/0075; A61F 2/246

(57) ABSTRACT

In some examples, the disclosure describes annuloplasty devices, systems, and methods involving one or more flexible elongated elements attached to one or more anchors secured proximate an annulus of a cardiac or vascular valve. In some examples one or more anchors are proximate a first side of a valve annulus. One or more flexible elements attached to the anchors are tightened to pull the first side closer to a second side of the annulus, thus reducing a dimension of the annulus. In some examples, an annuloplasty system includes an annuloplasty ring and one or more anchors configured to attach the ring proximate a valve annulus. The ring may include permanently deformable section that can be deformed after implantation to change a dimension of a corresponding valve annulus.

This application claims the benefit of U.S. Provisional Application Ser. No. 62/779,294, entitled “HEART VALVE REPAIR,” and filed on Dec. 13, 2018, the entire content of which are incorporated herein by reference.

### TECHNICAL FIELD

This disclosure relates to heart valve repair, such as mitral valve repair.

### BACKGROUND

Patient conditions associated with heart valves can produce valvular insufficiency or regurgitation. Valvular insufficiency or regurgitation occurs when a valve in a heart of a subject does not close completely, allowing blood to flow backwards (e.g., from the left ventricle to the left atrium), which may adversely impact the functionality of the heart.

The mitral valve includes two leaflets (anterior and posterior) attached to an annulus (e.g., a fibrous ring). In a healthy heart, the mitral valve leaflets close during contraction of the left ventricle and prevent blood from flowing back into the left atrium. Mitral valve regurgitation is a condition in which the leaflets of a mitral valve of a subject do not coapt properly and, as a result, blood regurgitates back into the left atrium from the left ventricle. The regurgitation of blood back into the left atrium may result in a reduced ejection volume from the left ventricle, causing the heart of the subject to work relatively hard to supply the desirable volume of blood to the body. Mitral regurgitation may occur because of different patient conditions. For example, secondary mitral regurgitation, also referred to as functional mitral regurgitation, may occur when a left ventricle dilates and causes dilation of the mitral annulus of a subject.

### SUMMARY

Some aspects of this disclosure describe examples of annuloplasty devices, systems, and methods for treating and/or repairing a heart valve, including, but not limited to, a mitral valve. The annuloplasty devices, systems, and techniques may enable reduction in spacing between valve leaflets, may improve coaptation of the valve leaflets, and may help reduce valvular insufficiency or regurgitation. Some examples described herein employ a minimalistic approach to septal-lateral cinching, e.g., of the mitral valve, through the use of a transcatheter, trans-septal approach for deploying an annuloplasty device. In some examples, one or more flexible elongated elements are attached to one or more anchors proximate the lateral side of a valve annulus and then tightened to pull the lateral side of the annulus closer to the anterior side of the annulus, thus reducing the septal-lateral dimension of the annulus. In some examples, an annuloplasty system includes an annuloplasty ring with a permanently deformable section, that when deformed after implantation, changes the dimensions of a corresponding valve annulus.

In some examples, the disclosure is directed to an annuloplasty system that includes an elongated flexible element including a proximal portion and a distal portion. The annuloplasty system includes at least one anchor configured to secure the elongated flexible element proximate an annulus of a cardiac or vascular valve. The annuloplasty system also includes a closure device configured to close a delivery opening in a tissue wall and secure the proximal and distal portions of the elongated flexible element. The elongated flexible element and the at least one anchor are configured to be delivered to the cardiac or vascular valve through the delivery opening in the tissue wall. In some examples, securing the proximal and distal portions of the elongated flexible element with the closure device pulls the at least one anchor and a portion of the annulus toward the closure device, thereby decreasing a width of the annulus.

In some examples, the disclosure is directed to an annuloplasty system that includes an elongated flexible element comprising a proximal portion, a distal portion, and an intermediate portion between the proximal and distal portions, and first, second, and third anchors. The first anchor is configured to secure the proximal portion of the elongated flexible element to a first tissue site adjacent a cardiac or vascular valve annulus. The second anchor is configured to secure the distal portion of the elongated flexible element to a second tissue site adjacent the cardiac or vascular valve annulus. The third anchor is configured to secure the intermediate portion of the elongated flexible element proximate the valve annulus. At least one of the first, second, and third anchors includes a rotatable portion configured to receive and/or attach to a portion of the elongated flexible element. Turning the rotatable portion winds the portion of the elongated flexible element about the rotatable portion, thereby decreasing a length of the elongated flexible member between the first anchor and the third anchor, and/or decreasing a length of the elongated flexible member between the second anchor and the third anchor so as to pull the third anchor and a portion of the annulus toward the first and second anchors, thereby decreasing a width of the annulus.

In some examples, the disclosure is directed to an annuloplasty system that includes an elongated flexible element including a proximal portion and a distal portion. The annuloplasty system further includes a first anchor configured to secure the proximal portion of the elongated flexible element proximate a cardiac or vascular valve annulus on a first side of the valve. The annuloplasty system further includes a second anchor configured to secure the elongated flexible element proximate the valve annulus on a second side of the valve apart from the first side. The first and/or second anchor and/or the distal portion of the elongated flexible element are configured such that the distal portion of the elongated flexible element can pass through a portion of the first and/or second anchor in a first direction but cannot be retracted through the first and/or second anchor in an opposite second direction, such that pulling the flexible element through the first and/or second anchor shortens a distance between the first and second anchors, thereby decreasing a width of the annulus.

In some examples, the disclosure is directed to an annuloplasty system including an elongated flexible element, a first anchor and a second anchor. The elongated flexible element includes a proximal portion and a distal portion. The first anchor is configured to secure the proximal portion of the elongated flexible element at a first location about a cardiac or vascular valve annulus. The second anchor is configured to secure the elongated flexible element at a second location about the valve annulus apart from the first location. In some examples the first anchor and/or the distal portion of the elongated flexible element are configured such that the distal portion of the elongated flexible element can pass through a portion of the first anchor in a first direction but cannot be retracted through the first anchor in an opposite second direction, such that pulling the flexible element through the first anchor shortens a distance between the first and second anchors, thereby decreasing a width of the annulus.

In some examples, the disclosure is directed to an annuloplasty system including a first annuloplasty device and a second annuloplasty device, where each of the first and second annuloplasty devices includes an elongated flexible element, a first anchor, and a second anchor. The elongated flexible element includes a proximal portion and a distal portion. The first anchor is configured to secure the proximal portion of the elongated flexible element at a first location about a cardiac or vascular valve annulus. The second anchor is configured to secure the elongated flexible element at a second location about the valve annulus apart from the first location. In some examples, the first anchor and/or the distal portion of the elongated flexible element are configured such that the distal portion of the elongated flexible element can pass through a portion of the first anchor in a first direction but cannot be retracted through the first anchor in an opposite second direction, such that pulling the flexible element through the first anchor shortens a distance between the first and second anchors, thereby decreasing a width of annulus. In some examples, the first and second annuloplasty devices operate independently and are anchored to separate portions of the valve annulus.

In some examples, the disclosure is directed to an annuloplasty system including an annuloplasty ring and a plurality of movable joints. The annuloplasty ring includes first, second, and third ring portions, and a plurality of anchors configured to secure the first, second, and third portions proximate to a cardiac or vascular valve annulus. The movable joints join the third ring portion together with the first and second ring portions. The third ring portion includes a permanently deformable material. Deforming the third ring portion pulls together the first and second ring portions, thereby decreasing the diameter of the annuloplasty ring and a corresponding width of the valve annulus.

In some examples, the disclosure is directed to a method for repairing a cardiac or vascular valve. The method includes advancing a delivery device through vasculature of a patient to a treatment site such as, for example, a cardiac or vascular valve. The method also includes releasing an annuloplasty device from the delivery device. The annuloplasty device includes at least one anchor. The method also includes attaching the at least one anchor to tissue proximate to an annulus of the valve. The method also includes cinching the annuloplasty device to decrease a width of the valve annulus.

The details of one or more examples are set forth in the accompanying drawings and the description below. Other features, objects, and advantages of examples according to this disclosure will be apparent from the description and drawings, and from the claims.

### BRIEF DESCRIPTION OF THE DRAWINGS

FIGS. 1A and 1B are schematic cross-sectional views of an example human heart.

FIG. 2 is a schematic cross-sectional view of the example human heart of FIG. 1A depicting atrioventricular and semi-lunar valves.

FIG. 3A is a schematic cross-sectional view of an example human heart and an example annuloplasty device.

FIG. 3B is a partial cross-sectional view of the annuloplasty device of FIG. 3A.

FIG. 4 is a schematic cross-sectional view of a heart valve and an example annuloplasty device.

FIG. 5 is a schematic cross-sectional view of a heart valve and an example annuloplasty device.

FIG. 6 is a schematic cross-sectional view of a heart valve and an example annuloplasty device.

FIG. 7 is a schematic cross-sectional view of a heart valve and an example annuloplasty device.

FIG. 8 is a flow diagram illustrating an example method for implanting an example annuloplasty device.

FIG. 9 is a schematic cross-sectional view of an example delivery device for delivering an example annuloplasty device.

### DETAILED DESCRIPTION

This disclosure describes annuloplasty devices, systems, and techniques for repairing a heart valve, such as, but not limited to, a mitral valve.

The annuloplasty devices, systems, and techniques described herein generally may enable reduction in spacing between valve leaflets, may improve coaptation of the valve leaflets, and may help reduce valvular insufficiency or regurgitation. While examples of the disclosure are described primarily with regard to treatment of the mitral valve, treatment of other heart valves is also contemplated.

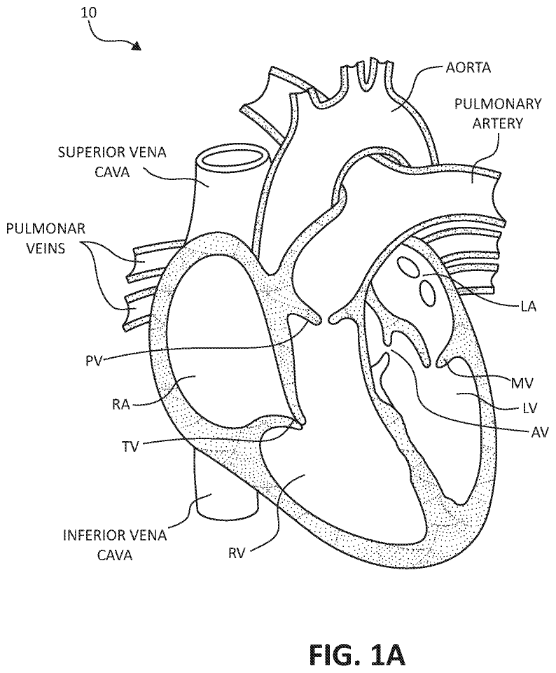

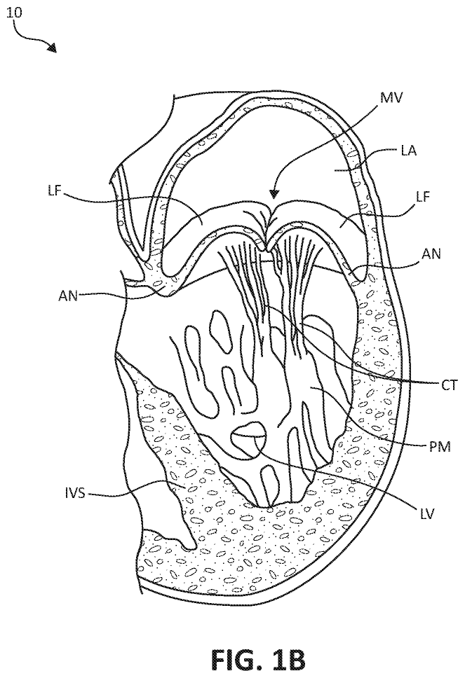

FIGS. 1A and 1B are schematic cross-sectional views of an example human heart 10. The human heart 10 is a four chambered, muscular organ that provides blood circulation through the body during a cardiac cycle. The four main chambers include the right atrium (RA) and right ventricle (RV) which supplies the pulmonary circulation, and the left atrium (LA) and left ventricle (LV) which supplies oxygenated blood received from the lungs to the remaining body. To ensure that blood flows in one direction through the heart, atrioventricular valves (tricuspid valve (TV) and mitral valves (MV)) are present between the junctions of the atrium and the ventricles, and semi-lunar valves (pulmonary valve (PV) and aortic valve (AV)) govern the exits of the ventricles leading to the lungs and the rest of the body. These valves contain leaflets (LF) or cusps that open and shut in response to blood pressure changes caused by the contraction and relaxation of the heart chambers. FIG. 1B is a schematic sectional illustration of a left ventricle LV of heart 10 showing anatomical structures and a native mitral valve MV.

The left atrium LA receives oxygenated blood from the lungs via the pulmonary veins and pumps the oxygenated blood through the mitral valve MV and into the left ventricle LV during ventricular diastole. The left ventricle LV contracts during systole and blood flows outwardly through the aortic valve AV, into the aorta and to the remainder of the body. In a healthy heart, the leaflets LF of the native mitral valve MV meet evenly at the free edges or “coapt” to close and prevent back flow of blood into the left atrium LA during contraction of the left ventricle LV. The tissue of the leaflets LF attach to the surrounding heart structure via a dense fibrous ring of connective tissue called an annulus AN. The flexible tissue of the leaflets LF of the native mitral valve MV are connected to papillary muscles PM, which extend upwardly from the lower wall of the left ventricle LV and the interventricular septum IVS, via branching tendons called chordae tendineae CT.

Mitral valve regurgitation is a condition in which the leaflets of a mitral valve of a subject do not coapt properly and, as a result, blood regurgitates back into the left atrium LA from the left ventricle LV. The regurgitation of blood back into the left atrium LA may result in a reduced ejection volume from the left ventricle LV, causing the heart of the subject to work relatively hard to supply the desirable volume of blood to the body. Mitral regurgitation may occur because of one or more patient conditions. For example, secondary mitral regurgitation, also referred to as functional mitral regurgitation, may occur when the left ventricle LV dilates and causes dilation of the mitral annulus of a subject. The leaflets LF of the valves may move apart as a result of the dilation of the left ventricle LV, which may adversely impact the ability of the leaflets to properly coapt.

In addition to or instead of being caused by dilation of the left ventricle LV, mitral valve regurgitation (or other valve regurgitation) may be caused by calcified plaque buildup in heart 10. For example, the leaflets LF of the valves (e.g., aortic valve AV or mitral valve MV) may harden and may not sufficiently coapt or meet, such that regurgitation may occur where the valve does not close completely, allowing blood to flow backwards (e.g., from the left ventricle LV to the left atrium LA). The left side of heart 10 (e.g., mitral valve MV and aortic valve AV) can be more likely to become calcified because of the higher pressures generated.

In some examples, heart 10 may suffer from Secondary Mitral Regurgitation, or Functional Mitral Regurgitation (FMR). Secondary Mitral Regurgitation or FMR may occur when a diseased left ventricle dilates and causes the dilation of the mitral annulus. This dilation does not allow the leaflets to coapt appropriately, and blood will be regurgitated back into the left atrium, causing the heart to work even harder to appropriately supply blood to the body.

In some examples, a surgical technique may be used to implant a ring on the annulus, referred to as annuloplasty. A goal of annuloplasty in FMR patients may be to reduce the distance between the two leaflets, or the septal-lateral annular diameter. Such open-heart surgery can be difficult for patients who are already very sick, and physicians are looking for a less invasive way to treat. The annuloplasty devices, systems, and techniques described herein may be used to repair a valve of heart 10 via a minimally invasive or relatively non-invasive medical procedure, e.g., via a transcatheter, trans-septal medical procedure that is less invasive than open heart surgery. While open heart surgeries, such as annuloplasty performed via open heart surgery, may have positive outcomes, a more minimally invasive medical procedure may be associated with a shorter recovery time for some patients.

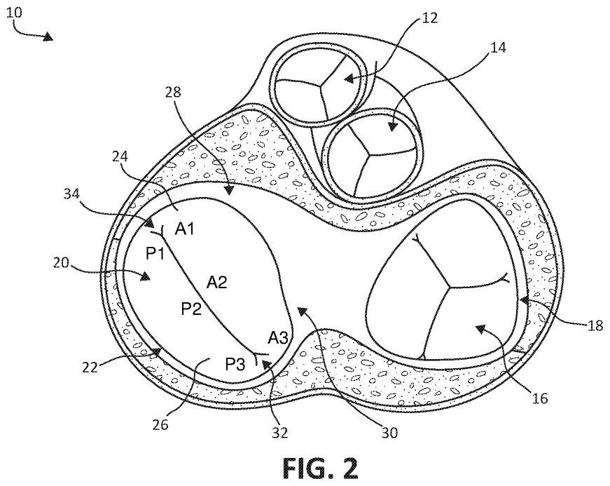

FIG. 2 is a schematic cross-sectional view of the example human heart 10 of FIG. 1A, illustrating the locations of the atrioventricular and semi-lunar valves. A pulmonary valve 12 and an aortic valve 14 govern the exits of the ventricles leading to the lungs and the rest of the body. A tricuspid valve 16 includes three leaflets attached to the surrounding heart tissue via a tricuspid valve annulus 18. A mitral valve 20 includes a mitral valve annulus 22. An anterior leaflet 24 and a posterior leaflet 26 are attached to the mitral valve annulus 22. The leaflets of the mitral valve 20, as well as the leaflets of other heart valves, are also referred to as cusps.

FIG. 2 also illustrates the general location of the left fibrous trigone 28 and right fibrous trigone 30 of heart 10. The posterior commissure 32 and the anterior commissure 34 of the mitral valve 20 indicate the areas where the mitral valve posterior leaflet 26 and anterior leaflet 24 come together.

In some examples an annuloplasty system is designed or configured to include multiple (e.g., 2, 3-5, or more) anchors around a lateral side of a valve annulus. In some examples the anchors can be screws, nitinol, or another type of anchoring system. The anchors are attached through an elongated flexible element such as, for example, a suture or wire. In an example using a wire, the ends of the wire are pulled tight, which pulls the lateral side of the annulus towards a delivery hole in the septum, thereby reducing the septal-lateral diameter of the annulus. The wire (or, e.g., suture) can then be locked in place with a closure device in the septum. The hole in the septum may in some circumstances be considered a potential risk for clinical outcomes. Accordingly, the closure device can in some examples serve two purposes: maintaining the cinch across the annulus in the appropriate direction, and closing the hole created in the septum for delivery of the system components.

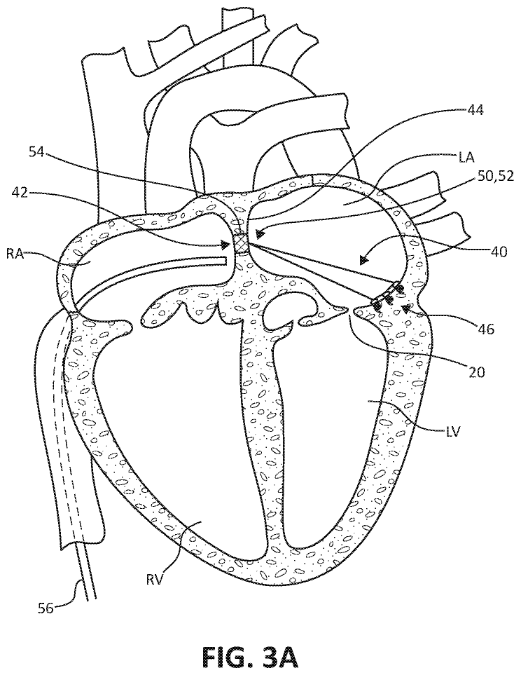

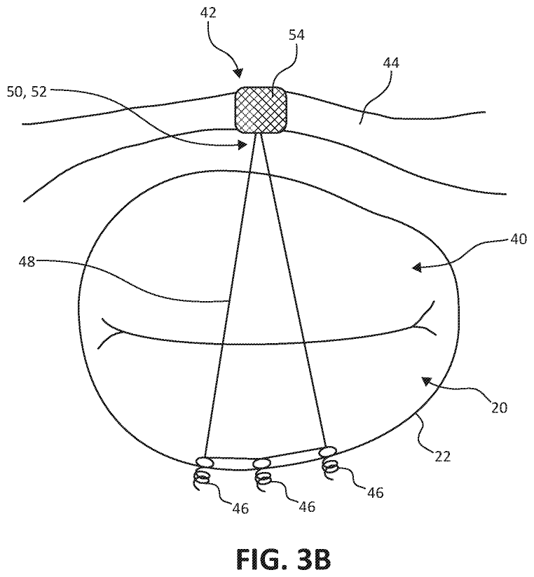

FIG. 3A is a schematic cross-sectional view of an example human heart, such as heart 10 described above, and example annuloplasty device 40. FIG. 3B is a partial horizontal, cross-sectional view of FIG. 3A looking down at annuloplasty device 40 implanted at the site of mitral valve 20. In this example annuloplasty device 40 is delivered into the left atrium LA through delivery hole 42 in the interatrial septum 44. Annuloplasty device 40 includes multiple anchors 46 that are implanted around the lateral side of the mitral annulus 22. Flexible elongated element 48 is attached to anchors 46. Ends 50, 52 of flexible elongated element 48 are attached to closure device 54 positioned in delivery hole 42 in interatrial septum 44.

As shown in FIGS. 3A and 3B, flexible elongated element 48 passes through a portion of each of anchors 46. During implantation of the annuloplasty device, anchors 46 are secured in the posterior or lateral side of mitral annulus 22. Ends 50, 52 of flexible element 48 are pulled toward the anterior side of annulus 22 in the direction of interatrial septum 44. This action pulls the lateral side of mitral annulus 22 toward interatrial septum 44 thereby reducing the septal-lateral diameter of annulus 22. In some cases, flexible element 48 slips through each of anchors 46 so that tension applied to the flexible element is distributed among the anchors.

After pulling the lateral side toward the atrial septum by a desired amount, ends 50, 52 of flexible element 48 are locked in place with closure device 54 placed in delivery hole 42 in septum 44. Accordingly, in the example shown in FIGS. 3A-3B, closure device 54 both maintains the cinching of mitral annulus 22 and closes delivery hole 42 previously created in interatrial septum 44. In some examples, a closure device can include a septal occlusion device configured to close an opening in a septum of the heart. For example, the closure device can include a frame or mesh structure positioned in and extending out about both sides the delivery opening and septal wall. In some examples a membrane material comprising, e.g., a polyester, polytetrafluoroethylene (PTFE), or other suitable material, is attached to the frame. In some examples the frame or mesh structure can be formed from a biocompatible material such as, for example, Nitinol.

Anchors 46 are configured to insert into the heart tissue and remain in place in the presence of the opposing force from flexible element 48. In some examples, anchors 46 each include a helix or double helix that is configured to be advanced into tissue of heart 10. For example, an anchor may be spirally advanced in the posterior, e.g., lateral, portion of annulus 22 and/or into posterior leaflet 26. The helix or double helix may optionally include an attachment, such as a hook, loop, or the like that is configured to receive and/or attach to flexible element 48 so that tension applied to flexible element 48 acts on the anchor and the surrounding tissue. In some examples, anchors 46 are formed as screws and/or may include a biocompatible metal or alloy, such as nitinol, stainless steel, a cobalt-chromium alloy, or the like. In some examples, anchors 46 can include features similar to one or more features provided by the implant system available from Medtronic, Inc., Minneapolis, Minn., under the name Heli-FX™ EndoAnchor™.

The example illustrated in FIGS. 3A-3B depicts the use of three anchors 46, but any number of anchors may be used depending upon the particular circumstances. For example, in some cases the annuloplasty device may include at least one anchor, at least two anchors, at least three anchors, or more than three anchors.

Elongated flexible element 48 is configured to be deployed and remain within example heart 10, and accordingly includes a suitable biocompatible material. In some examples, flexible element 48 includes a suture or a wire configured to cinch mitral annulus 22. Some examples of possible materials and configurations for flexible element 48 include a monofilament, a braid of a plurality of filaments of the same or material or of filaments from different materials, a braided sheath with a single filament core, and/or a braided sheath with a braided core. In some cases, the flexible element may be composed of a biocompatible material such as, but not limited to, nylon or polyester. In some examples, a flexible element can be formed at least in part from a material that does not stretch. In some cases, a flexible element may be pre-stressed to prevent the flexible element from elongating after the annuloplasty device 40 is implanted. One example of a suitable material includes a pre-stretched ultra-high-molecular-weight polyethylene.

FIG. 3A also illustrates a delivery device 56 for the annuloplasty device 40. In the example of FIG. 3A, the delivery device 56 includes a catheter. The catheter may define an internal lumen that extends from proximate a proximal end of the catheter to proximate a distal end of the catheter (e.g., may extend from the proximal end to the distal end). The lumen may be configured to house the annuloplasty device 40 during percutaneous introduction of the catheter into vasculature of a patient and advancing of the distal end of the catheter to the treatment location.

In some examples, the catheter may be used with a guidewire, a guide catheter, or the like, to facilitate introduction of the catheter into vasculature of a patient and advancing of the distal end of the catheter to the treatment location. In some examples, the catheter includes a steerable shaft and/or distal tip to allow a clinician to control positioning of the distal tip relative to anatomical structures, such as heart 10. In some examples, a delivery system may include a steerable guide and/or a catheter-based torque member similar to the steerable guide and/or application device provided with the implant system available from Medtronic, Inc., Minneapolis, Minn., under the name Heli-FX™ EndoAnchor™.

In some examples, to facilitate positioning of the delivery device, e.g., the catheter, the annuloplasty device 40, or both, within the treatment location, a distal portion of the catheter may include at least one radiographic marker configured to be visualized using a radiographic technique.

In some examples, the catheter may access the left atrium LA trans-septally. For example, as shown in FIG. 3A, annuloplasty device 40 is delivered to the left atrium LA by inserting the catheter through the inferior vena cava into the right atrium RA and then through the delivery hole 42 in the interatrial septum 44. It is appreciated that this is only one of many possible methods of deployment and treatment sites.

Delivery device 56 (e.g., the catheter of FIG. 3A) is also configured to deploy annuloplasty device 40 in a position proximate mitral annulus 22 (e.g., FIG. 3B). For example, the delivery device can be configured to advance one or more of the anchors 46 to the lateral side of mitral annulus 22 and then insert the anchor(s) into the heart tissue within or proximate to annulus 22 as shown in FIG. 3B. In some examples flexible element 48 may be pre-attached to one or more anchors 46 within the delivery device. Retracting the delivery device from the lateral or posterior side of annulus 22 may pull the ends 50, 52 of flexible element 48 toward the delivery opening 42. Flexible element 48 can then be drawn taut to pull the lateral side of annulus 22 toward the delivery opening by a desired amount. The ends of flexible element 48 may then be secured to closure device 54, which can be attached to the interatrial septum 44 within the delivery opening 42 to close the opening.

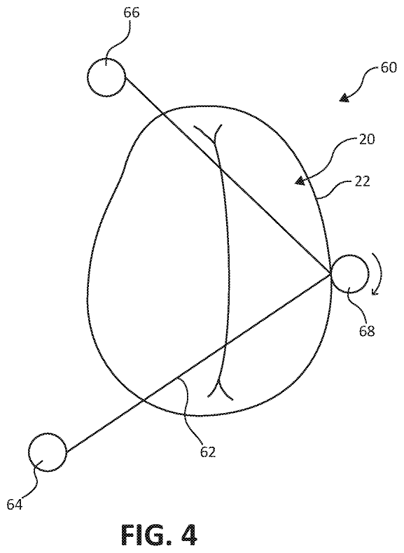

While the description of a delivery system has been provided above with reference to delivery device 56 in FIG. 3A, it is understood that the devices, systems, techniques, components, and other aspects described can also be applicable to the other examples of annuloplasty systems, devices, and methods provided herein, including those examples explained with reference to FIGS. 4-9.

FIG. 4 is a schematic cross-sectional view of a heart valve and another example annuloplasty device 60. In this example the heart valve is depicted as mitral valve 20 with mitral annulus 22, though it should be understood that other possible treatment sites, including other heart valves, are contemplated. As shown in FIG. 4, the annuloplasty device 60 includes at least three anchors connected by an elongated flexible element 62. In some examples the three anchors are implanted proximate annulus 22 on different sides or areas of annulus 22. For example, in FIG. 4, first anchor 64 is implanted in the left fibrous trigone and second anchor 66 is secured in the right fibrous trigone. Third anchor 68 is secured in the heart tissue in the P2 region of the posterior annulus. The ends of flexible element 62 are attached to the first and second anchors 64, 66. An intermediate portion of flexible element 62 is secured to and/or wrapped about the third anchor 68. It is understood that other locations and/or regions of implantation are possible for some or all of the anchors.

In some examples, one of the first, second, and third anchors can be configured to adjustably secure a portion of flexible element 62. For example, the third anchor 68 can be configured to adjustably secure flexible element 62. In some examples, the third anchor 68 may include a first portion, such as a screw or tine, that is inserted into the heart tissue. A second portion of the anchor 68 may swivel or turn with respect to the first portion (e.g., as indicated by the arrow at anchor 68). During deployment, flexible element 62 becomes tighter, effectively shortening as it winds around the turning second portion of third anchor 68. The tightening and shortening flexible element 62 cinches the mitral annulus as shown in FIG. 4 by pulling the posterior annulus toward the trigones. In some cases, the second portion of the third anchor can be pushed down into the first portion or other manipulated to rotationally lock the second portion and the flexible element in place.

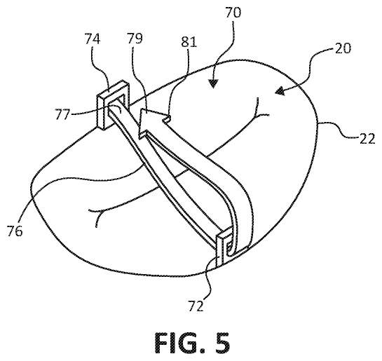

FIG. 5 is a schematic cross-sectional view of a heart valve and another example annuloplasty device 70. In this example, annuloplasty device 70 includes first anchor 74 implanted at a first location on a first side of annulus 22. Second anchor 72 of annuloplasty device 70 is implanted or secured at a second location on a second side of annulus 22, apart from the first side and location. Flexible element 76 connects first anchor 74 and second anchor 72. In some examples, a proximal portion, e.g., one end, of flexible element 76 is connected to one of the anchors and a distal portion, e.g., the other end, of the flexible element wraps around or through the other anchor and is then secured at the beginning anchor. For example, FIG. 5 illustrates the case in which flexible element 76 is attached to first anchor 74 at a proximal portion being first end 77. A distal portion, in this case second end 79, of flexible element 76 passes through a loop portion of second anchor 72 and is then directed back to first anchor 74, to which it can be secured. In some cases, the flexible element passes through a loop portion of first anchor 74 such that the flexible element cannot be retracted back through the second anchor. In some examples flexible element cannot be retracted back through the loop portion of second anchor 72.

In some examples, the anchors and flexible element shown in FIG. 5 can be used to decrease a dimension of a valve annulus at one or more locations about a valve annulus. In some examples involving a valve, one anchor is implanted proximate an anterior zone of the valve annulus and another anchor is implanted proximate a posterior zone of the valve annulus. For example, first anchor 74 can be secured proximate the A2 location of the mitral valve (FIG. 2) and second anchor 72 can be implanted proximate the P2 location of the mitral valve. In some examples first anchor 74 can be secured proximate the A1 location of the mitral valve and second anchor 72 can be implanted proximate the P1 location of the mitral valve. In some examples, first anchor 74 can be secured proximate the A3 location of the mitral valve and second anchor 72 can be implanted proximate the P3 location of the mitral valve. Other locations about a valve are also possible.

In some examples an annuloplasty system includes one, two, three, or more sets of flexible elements and anchors that can be implanted at multiple locations about a cardiac or vascular valve. Using one, two or more sets of flexible elements and anchors can enable a further customized treatment that may depend on, e.g., a specific patient anatomy and/or diametric reduction need. In some examples two or more flexible elements in parallel with each other across the valve in, e.g., a septal-lateral or anterior/posterior direction.

In the example shown in FIG. 5, flexible element 76 comprises a band formed from a biocompatible material. First anchor 74 and second end 79 of flexible element 76 are configured so that second end 79 can be advanced through in a first direction but cannot then be retracted or moved backward through first anchor 74. As an example, first anchor 74 and second end 70 can form a detent or catch system. The example in FIG. 5 illustrates second end 79 of flexible element 76 having an arrow shape with side protrusions 81 that catch on first anchor 74 when flexible element 76 is moved backward through the anchor. Other types of catch or detent systems can also be used. In some examples second anchor 72 can also be configured to prevent backward movement of flexible element 76 through the loop portion of anchor 72. The locking and/or ratcheting mechanism provided by the configuration of one or more of first anchor 74, second anchor 72, and second end 79 of flexible element 76 can lock the flexible element or band in place to help maintain the cinch across the septal-lateral direction of annulus 22.

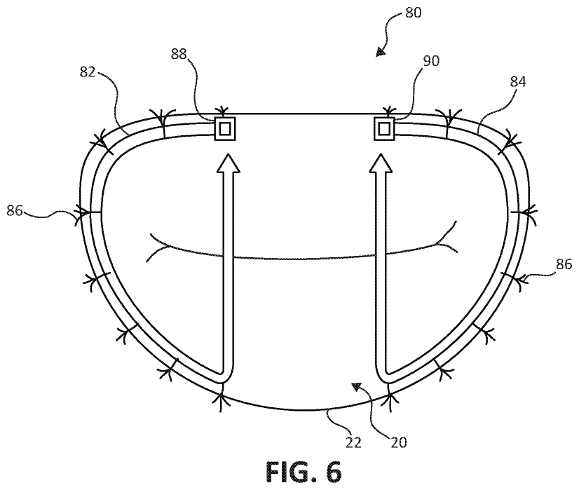

FIG. 6 is a schematic cross-sectional view of a heart valve and another example annuloplasty device 80. The annuloplasty device 80 includes first and second flexible elements 82, 84 that are separately anchored around separate portions of the mitral valve 20 and annulus 22. In some cases, each of flexible elements 82, 84 are configured as flexible element 76 described with respect to FIG. 5. As shown in FIG. 6, the first flexible element 82 is configured as a band that is secured about one side of the mitral valve 20 proximate the mitral annulus 22. The second flexible element 84 is also configured as a band and is secured about the opposite side of the mitral valve 20 proximate the mitral annulus 22. A plurality of suitable anchors 86 attach the first and second flexible elements 82, 84 to the heart tissue proximate annulus 22.

The first flexible element 82 includes a first cinching anchor 88 and the second flexible element 84 includes a second cinching anchor 90. As with the example illustrated in FIG. 5, in this case distal portions, e.g., the ends, of flexible elements 82, 84 can be inserted into a loop portion of the respective anchor 88, 90. The anchors 88, 90 are configured to accept insertion of the flexible elements in a first direction but resist movement of the inserted flexible elements in the opposite direction. Thus, the ends of flexible elements 82, 84 are inserted and pulled through the first and second anchors 88, 90, respectively, to tighten the bands and cinch the mitral annulus. The separate nature of the first and second flexible elements in this example can enable cinching of different parts of annulus 22 by different amounts.

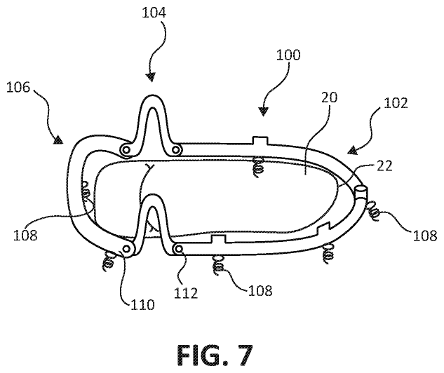

FIG. 7 is a schematic cross-sectional view of a heart valve and another example annuloplasty device 100. The annuloplasty device 100 is configured as a ring having multiple connected sections. Multiple anchors 108 secure the ring proximate the annulus of the valve. As shown in FIG. 7, the annuloplasty device 100 includes first and second portions 102, 106 that are joined by a third portion 104. In some cases, the third portion 104 is attached to the first and second portions 102, 106 with movable joints 110, 112, such as hinges or rivets.

Some examples of the annuloplasty device 100 are configured to re-dimension the device ring by deforming a portion of the ring. As an example, in FIG. 7 the third portion 104 is formed from a mechanically, permanently deformable material. One example of a possible material is a deformable metal such as, for example, MP35N. The first and second portions 102, 106 of the ring comprise a flexible metal in this case, which conforms to the contours of the annulus. Examples of possible materials for the first and second portions includes Nitinol and the like. Once secured about the annulus, the third portion 104 of the device 100 can be crimped to bring the first and third portions 102, 106 closer together, thereby reducing the septal-lateral dimension of the valve. The movable (e.g., hinging or riveting) nature of the joints between the second portion 106 and the first and third portions 102, 104 of the device can allow independent movement of the anterior and posterior sections of the valve, thus potentially facilitating movement that is closer to the inherent anatomical movement.

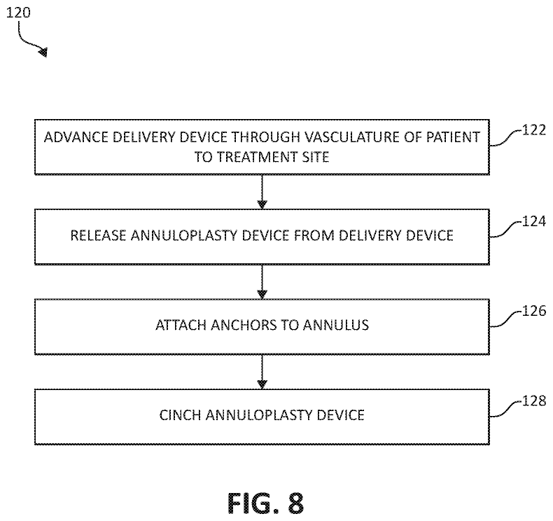

FIG. 8 is a flow diagram illustrating a method 120 for implanting an annuloplasty device, such as, but not limited to, one of the devices described herein. For example, the technique of FIG. 8 is described with concurrent reference to annuloplasty device 40 of FIGS. 3A and 3B, although it will be understood that the technique of FIG. 8 may be used to implant any of the annuloplasty devices described herein, and the annuloplasty devices described herein may be implanted using other techniques.

Delivery device 56 may be advanced through vasculature of a patient of a treatment site (122). For example, a clinician may introduce delivery device 56 into vasculature of a patient transcutaneously. For instance, the delivery device 56 may be introduced to a femoral or radial artery. The delivery device 56 may be advanced through vasculature of the patient to the treatment site by a clinician manipulating a handle of delivery device 56. In some examples, the delivery device 56 may include a steerable shaft or tip to allow the clinician to direct delivery device 56 through bends, curves, and branching points of the vasculature.

As shown in FIG. 3A, the delivery device is directed through the inferior vena cava into the right atrium RA and then through the delivery hole 42 in the interatrial septum 44. In some examples, the treatment site may include the mitral valve, and the delivery device 56 may be advanced to the left atrium. In other examples, the treatment site may include another heart valve. The delivery device 56 may access the left atrium trans-septally, trans-aortically, or trans-apically. In some examples, the delivery device 56 may be tracked over a guide wire, through a guide catheter, or the like as the delivery device 56 is advanced to the treatment site. The delivery device 56 may include one or more radiological markers at or near a distal end of the delivery device 56 to assist visualizing the delivery device 56 as delivery device is advanced to the treatment site.

Once the delivery device 56 (e.g., a distal portion of the delivery device 56) has been advanced to the treatment site, the delivery device 56 may release the annuloplasty device 40, including flexible element 48 and the anchors 46 (124). The particular way in which the annuloplasty device 40 is released by the delivery device 56 may depend on the configuration of the annuloplasty device 40. Releasing the annuloplasty device 40 may include, for example, moving the anchors 46 between an undeployed configuration in which the anchors extends generally inward into the catheter and a deployed configuration in which the anchors extend generally outward away from the catheter.

After releasing the annuloplasty device 40, the anchors 46 are attached to the lateral or posterior side of the annulus, e.g., in the configuration shown in FIGS. 3A and 3B (126). Following attachment, the ends 50, 52 of flexible element 48 are pulled toward the anterior side of annulus 22 in the direction of the interatrial septum 44 and locked in place with the closure device 54 placed in the delivery hole 42 in the septum 44. These actions pull the lateral side of the mitral annulus 22 toward the interatrial septum 44 and maintain its position, thereby reducing the septal-lateral diameter of annulus 22, e.g., cinching the annuloplasty device 40 and annulus 22.

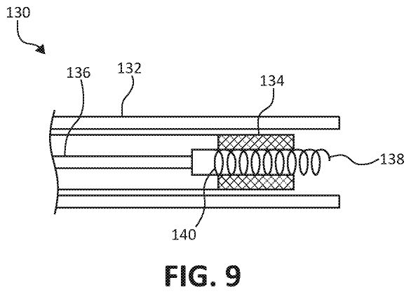

After implanting first anchor 138, the delivery system moves second anchor 140 to another site for deployment. Although two anchors are shown in FIG. 9, three or more anchors could be deployed in this fashion. In some examples, multiple anchors can also be delivered one at a time.

FIG. 9 is a side cross-sectional view of a distal portion of an example delivery device. In this example delivery device 130 includes steerable shaft 132, which allows a clinician to direct delivery device 130 through bends, curves, and branching points of the vasculature. Delivery device 130 includes retaining sleeve 134 positioned at the tip of torque member 136. In this example, multiple anchors are stacked on top of each other in the delivery system. For example, in FIG. 9, first anchor 138 and second anchor 140 are positioned inside retaining sleeve 134 and attached to torque member 136. In some examples, first anchor 138 is attached to second anchor 140, such that during deployment, when second anchor 140 is rotated by torque member 136, first anchor 138 will be torqued and deployed into a tissue site such as a valve annulus. In some examples elongated flexible element 142, e.g., a cinch wire, may be included in the delivery device and deployed at the same time.

Various examples have been described. These and other examples are within the scope of the following claims.

What is claimed is:

1. An annuloplasty system comprising:
an elongated flexible element comprising a proximal portion and a distal portion;
at least one anchor configured to secure the elongated flexible element proximate an annulus of a cardiac or vascular valve; and
a closure device configured to close a delivery opening in a tissue wall and secure the proximal and distal portions of the elongated flexible element.

2. The annuloplasty system of claim 1, wherein the elongated flexible element and the at least one anchor are configured to be delivered to the cardiac or vascular valve through the delivery opening in the tissue wall.

3. The annuloplasty system of claim 1, wherein the closure device, when secured to the elongated flexible element, pulls the at least one anchor and a portion of the annulus toward the closure device, to decrease a width of the annulus.

4. An annuloplasty system comprising:
an elongated flexible element comprising a proximal portion, a distal portion, and an intermediate portion between the proximal and distal portions;
a first anchor configured to secure the proximal portion of the elongated flexible element to a first tissue site adjacent a cardiac or vascular valve annulus;
a second anchor configured to secure the distal portion of the elongated flexible element to a second tissue site adjacent the cardiac or vascular valve annulus; and
a third anchor configured to secure the intermediate portion of the elongated flexible element proximate the valve annulus.

5. The annuloplasty system of claim 4, wherein at least one of the first, second, and third anchors comprises a rotatable portion configured to receive and attach to a portion of the elongated flexible element.

6. The annuloplasty system of claim 5, wherein turning the rotatable portion winds the portion of the elongated flexible element about the rotatable portion to at least one of decrease a length of the elongated flexible member between at least one of the first anchor and the third anchor or the second anchor and the third anchor to pull the third anchor and a portion of the annulus toward the first anchor and the second anchors to decrease a width of the annulus.

7. An annuloplasty system comprising:
an elongated flexible element comprising a proximal portion and a distal portion;
a first anchor configured to secure the proximal portion of the elongated flexible element proximate a cardiac or vascular valve annulus on a first side of the valve; and
a second anchor configured to secure the elongated flexible element proximate the valve annulus on a second side of the valve apart from the first side;
wherein at least one of the first anchor or the second anchor is configured such that the distal portion of the elongated flexible element can pass therethrough in a first direction and cannot be retracted therethrough in a second direction opposite the first direction.

8. The annuloplasty system of claim 7, wherein pulling the elongate flexible element through the first anchor or the second anchor shortens a distance between the first and second anchors, thereby decreasing a width of the annulus.

9. The annuloplasty system of claim 7, wherein the elongate flexible element comprises a first elongate flexible element, and wherein the annuloplasty system further comprises:
a second elongated flexible element comprising a proximal portion and a distal portion;
a third anchor configured to secure the proximal portion of the second elongated flexible element at a third location about a cardiac or vascular valve annulus; and
a fourth anchor configured to secure the elongated flexible element at a fourth location about the valve annulus apart from the third location;
wherein at least one of the third anchor or the fourth anchor is configured such that the distal portion of the elongated flexible element can pass therethrough in a first direction and cannot be retracted therethrough in a second direction opposite the first direction.

10. The annuloplasty system of claim 7, wherein the first location is different from the third location and the fourth location, and wherein the second location is different from the third location and the fourth location.

11. The annuloplasty system of claim 7, wherein the first elongate flexible element is movable independent of the second elongate flexible element.

12. The annuloplasty system of claim 7, further comprising at least one deformable segment coupled to the first elongate flexible element and the second elongate flexible element, wherein the deformable segment comprises a preformed shape configured to urge the first elongate flexible element toward the second elongate flexible element.

13. A method for repairing a cardiac or vascular valve, the method comprising:
advancing a delivery device through vasculature of a patient to a treatment site comprising a cardiac or vascular valve;
releasing an annuloplasty device from the delivery device, the annuloplasty device comprising at least one anchor;
attaching the at least one anchor to tissue proximate to an annulus of the valve; and
cinching the annuloplasty device to decrease a width of the valve annulus.

14. The method of claim 13, wherein advancing the delivery device comprises advancing the delivery device through a septum of a heart or through an apex of the heart.

15. The method of claim 13, wherein the delivery device comprises at least one radiopaque marker, and wherein advancing the delivery device comprises visualizing the delivery device via fluoroscopy.

16. The method of claim 13, wherein releasing the annuloplasty device comprises controlling the at least one anchor between an undeployed configuration, in which the at least one anchor extends generally inward into a lumen of the delivery device, and a deployed configuration, in which the at least one anchor extends generally outward away from the delivery device.

17. The method of claim 13, wherein the tissue comprises at least one of a lateral side of a mitral valve annulus or a posterior side of a mitral valve annulus.

18. The method of claim 13, wherein cinching the annuloplasty device comprises pulling the proximal portion and the distal portion of the elongated flexible element toward a closure device engaged with a septum of a heart and securing the proximal portion and the distal portion of the elongated flexible element to the closure device.

19. The method of claim 13, wherein the at least one anchor comprises a rotatable portion configured to receive a portion of the elongated flexible element, and wherein cinching the annuloplasty device comprises turning the rotatable portion to wind the elongated flexible element about the rotatable portion.

20. The method of claim 13, wherein the at least one anchor comprises a first anchor and a second anchor, and wherein cinching the annuloplasty device comprises drawing the elongate flexible member through both the first anchor and the second anchor to urge the first anchor toward the second anchor.
